(19) United States
(12) Patent Application Publication
(10) Pub. No.: US 2024/0287433 A1
(43) Pub. Date: Aug. 29, 2024

(54) CELL-CULTURE PLATFORM AND A MAGNETIC MATERIAL FOR SUCH A PLATFORM

(71) Applicants: National University of Singapore, Singapore (SG); Rexadvance Technology Pte. Ltd., Singapore (SG)

(72) Inventors: Boon Chuan LOW, Singapore (SG); Somsubhro MUKHERJEE, Singapore (SG); Qing LI, Singapore (SG); Ying Ming, Ivan YOW, Singapore (SG)

(21) Appl. No.: 18/289,923

(22) PCT Filed: May 27, 2022
(86) PCT No.: PCT/SG2022/050358

§ 371 Date: Nov. 8, 2023

(30) Foreign Application Priority Data
May 28, 2021 (SG) 10202105680V

(51) Int. Cl. C12M 1/42; C12M 1/00; C12N 5/00; C12N 13/00

(52) U.S. Cl. CPC C12M 35/06; C12M 23/22; C12N 5/0068; C12N 13/00

(57) ABSTRACT

A magnetic material for a cell-culture platform is disclosed herein. In a specific embodiment, the magnetic material comprises an elastomeric material and a dispersion of a plurality of magnetic particles embedded in the elastomeric material. A cell-culture platform including the magnetic material and a method of fabricating the magnetic material are also disclosed.

### CROSS-REFERENCE TO RELATED APPLICATIONS

The present application is a national phase entry under 35 U.S.C. § 371 of International Application No. PCT/SG2022/050358, filed May 27, 2022, published as International Publication No. WO 2022/250614 A2, which claims the benefit of Singapore Patent Application No. 10202105680V, filed May 28, 2021, the disclosures of all of which are hereby incorporated by reference in their entirety.

### BACKGROUND AND FIELD

The present disclosure relates to a cell culture platform and a magnetic material for such a platform, particularly but not exclusively, for use in a cell-stretching system.

Cells in-vivo undergo a variety of deformations caused by mechanical stresses in the body. For example, cells of connective, muscle and epithelial tissues are particularly susceptible to deformations as a consequence of their function.

In vitro cell-culture is employed extensively by researchers in order to study living tissues. However, the ability to replicate in-vivo conditions giving rise to cell deformation has so far been limited, with many solutions being complex and limited in the amount of deformation they can achieve.

It is desirable to provide a platform for cell culture which addresses at least one of the drawbacks of the prior art and/or to provide the public with a useful choice.

### SUMMARY

In a first aspect, there is provided a magnetic material for a cell-culture platform. The magnetic material comprises an elastomeric material and a dispersion of a plurality of magnetic particles embedded in the elastomeric material. Dispersing magnetic particles through an elastomer may enable a material which is magnetically deformable to be provided, without compromising on the high elasticity of the original elastomer.

The elastomeric material may comprise silicone rubber which may enable biocompatibility of the magnetic material and covey high elasticity.

The magnetic material may have an elongation at break of at least about 100%, particularly from about 200% to about 400%, such as about 250% or about 350%.

The dispersion of the plurality of magnetic particles may comprise a substantially homogenous, or even, distribution of magnetic particles within the elastomeric material, thereby enabling magnetic deformation of the magnetic material in any direction.

The magnetic particles may include magnetic-field inducible magnets. The magnetic particles may include filings, particularly iron filings.

The magnetic particles may include one or more of Iron, Cobalt, Nickel, Gadolinium, Dysprosium, Permalloy and Wairakite, or other ferromagnetic materials, particularly iron.

The weight of the magnetic particles may range from about 40% to about 80% of the weight of the elastomeric material, particularly from about 55% to about 65%, such as about 60% of the weight of the elastomeric material, which may provide a good balance between magnetic deformability and elasticity of the magnetic material.

In a second aspect, there is provided a cell-culture platform comprising: a biocompatible deformable receptacle for cell culture media; and a deformation actuator attached to the biocompatible deformable receptacle, the deformation actuator comprising an elastomeric material and a dispersion of a plurality of magnetic particles embedded in the elastomeric material, and being operable to deform the biocompatible deformable receptacle in response to a magnetic field.

Employing a deformation actuator comprising an elastomeric material and a dispersion of a plurality of magnetic particles embedded in the elastomeric material may enable the deformation actuator to be fabricated with high elasticity which in turn may enable large deformations of the deformable receptacle and consequently any cells contained within the deformable receptacle to be achieved. Further, precise control of the direction of the deformation may be possible due to the dispersion of the plurality of magnetic particles.

The biocompatible deformable receptacle may comprise a further elastomeric material, and the deformation actuator and the biocompatible deformable receptacle may be integrally formed as a composite material. The further elastomeric material may comprise silicone rubber.

The deformation actuator may enclose the biocompatible deformable receptacle and may therefore be operable to deform the biocompatible deformable receptacle along a plurality of axes.

The biocompatible deformable receptacle may comprise a base which is substantially transparent, thereby enabling straightforward imaging of cells cultured within the biocompatible deformable receptacle.

The biocompatible deformable receptacle may contain cells and/or cell-culture media.

In a third aspect, there is provided a method of producing a magnetic material. The method includes the steps of mixing a predetermined weight of magnetic particles into a quantity of a liquid elastomer to disperse the magnetic particles in the quantity of the liquid elastomer; and curing the quantity of the liquid elastomer to form the magnetic material.

The liquid elastomer may comprise silicone rubber. The magnetic particles may include filings, such as iron filings. The magnetic particles may include one or more of Iron, Cobalt, Nickel, Gadolinium, Dysprosium, Permalloy and Wairakite, and particularly Iron.

Mixing the predetermined weight of the magnetic particles into the quantity of the liquid elastomer may include mixing a weight of the iron filings amounting to from about 40% to about 80% of a weight of the quantity of the liquid elastomer, particularly from about 55% to about 65% such as about 60% of the weight of the quantity of the liquid elastomer. Mixing the predetermined weight of magnetic particles into the quantity of the liquid elastomer may include mixing the predetermined weight of magnetic particles into the quantity of liquid elastomer until a substantially homogenous distribution of the magnetic particles in the liquid elastomer is obtained.

In a fourth aspect, there is provided a method of producing a cell-culture platform, comprising: mixing a predetermined weight of magnetic particles into a quantity of a liquid elastomer to disperse the magnetic particles in the quantity of liquid elastomer; curing the quantity of the liquid elastomer to form a magnetic material; and attaching a biocompatible deformable receptacle for cell culture media to the formed magnetic material.

Attaching the biocompatible deformable receptacle for cell culture media to the magnetic material may include applying a further quantity of a further liquid elastomer to the magnetic material and curing the further quantity of the further liquid elastomer to form a composite material comprising the biocompatible deformable receptacle and the magnetic material. The further liquid elastomer may include silicone rubber. Applying the further quantity of the further liquid elastomer to the magnetic material may include removing a portion of the magnetic material to form a through-hole in the magnetic material; and introducing the further quantity of the further liquid elastomer into the through-hole.

In a fifth aspect, there is provided a method of deforming cells using a cell-culture platform, the cell-culture platform comprising: a biocompatible deformable receptacle for cell culture media; and a deformation actuator attached to the biocompatible deformable receptacle, the deformation actuator comprising an elastomeric material and a dispersion of a plurality of magnetic particles embedded in the elastomeric material, the method comprising: applying a cell-binding coating to an inner surface of the biocompatible deformable receptacle; seeding one or more cells onto the cell-adhesive; and actuating the dispersion of the plurality of magnetic particles using an applied magnetic field to deform the biocompatible deformable receptacle and the one or more cells. An axis of deformation of the one or more cells may be adjusted by adjusting an angle of the applied magnetic field relative to the cell-culture platform.

It is envisaged that features relating to one aspect may be applicable to the other aspects.

### BRIEF DESCRIPTION OF THE DRAWINGS

An exemplary embodiment will now be described with reference to the accompanying drawings, in which:

FIG. 1 illustrates a system for cell deformation according to a preferred embodiment;

FIG. 2a is a photograph of a cell-culture platform for inclusion in the system of FIG. 1;

FIG. 2b is a schematic illustration of a cross section of the cell-culture platform of FIG. 2a;

FIG. 3a illustrates a method of stretching and/or compressing cells using the system for cell deformation of FIG. 1;

FIG. 3b shows a schematic illustration of the deformation of the cell-culture platform of FIGS. 2a and 2b resulting from the method of FIG. 3a;

FIG. 4 illustrates a fabrication method of the cell-culture platform according to the embodiment of FIGS. 2a and 2b;

FIG. 5 is an image of a cell cultured in a prototype cell-culture platform according to the embodiment of FIGS. 2a and 2b;

FIG. 6 is a photograph illustrating manual stretching of the prototype cell-culture platform;

FIG. 7 is a series of four photographs illustrating deformation of the prototype cell-culture platform using a magnet;

FIGS. 8a to 8d show images of Hela cells cultured on the prototype cell-culture platform before and after stretching;

FIG. 9 shows a change in tensile stress as a function of tensile strain of two prototype cell-culture platforms according to the embodiment of FIGS. 2a and 2b;

FIG. 10 shows a change in tensile stress as a function of tensile strain of prototype cell-culture platforms according to the embodiment of FIGS. 2a and 2b at a range of experimentally relevant temperatures;

FIG. 11a shows the maximum distance between a magnet and the prototype cell-culture platforms employed to obtain to the results of FIG. 10 in order to achieve any deformation of the respective cell culture platform along its long axis;

FIG. 11b shows the maximum distance between a magnet and the prototype cell-culture platforms employed to obtain to the results of FIG. 10 in order to achieve any deformation of the respective cell culture platform along its short axis;

FIG. 12a shows the maximum distance between a magnet and the prototype cell-culture platforms employed to obtain to the results of FIG. 10 in order to achieve total deformation of the respective cell culture platform along its long axis;

FIG. 12b shows the maximum distance between a magnet and the prototype cell-culture platforms employed to obtain to the results of FIG. 10 in order to achieve total deformation of the respective cell culture platform along its short axis;

FIG. 13 illustrates a scatter plot of YAP Nucleus/Cytoplasmic ratio for cells within a monolayer tissue of mammalian cell line MDCK expressing YAP-GFP seeded within a prototype cell-culture platform according to the embodiment of FIGS. 2a and 2b before and after 4 hours of stretching;

FIGS. 14a to 14c are photographs of three cell-culture platforms according to alternative embodiments, respectively; and

FIGS. 15a to 15c are photographs of three prototype deformation actuator layers for cell-culture platforms according embodiments produced with the use of a mould.

### DETAILED DESCRIPTION OF PREFERRED EMBODIMENT

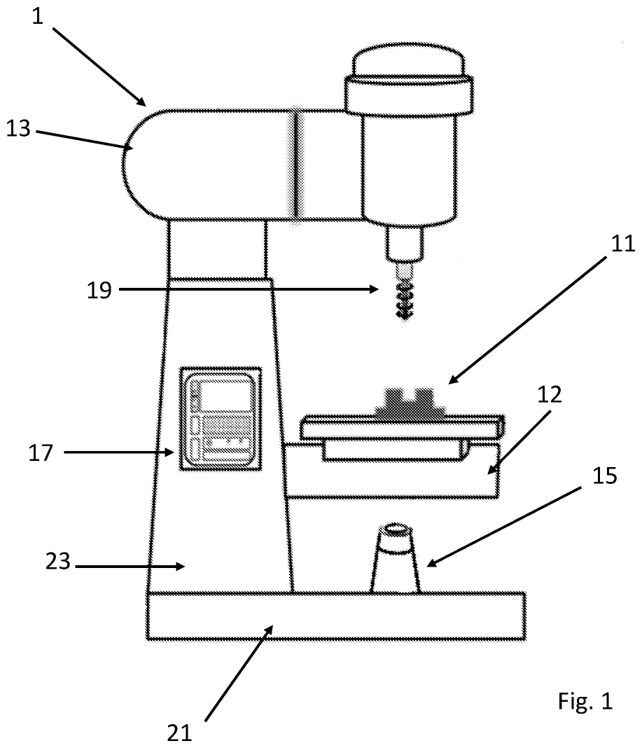

FIG. 1 illustrates a system 1 for cell deformation according to a preferred embodiment. The system 1 includes a base 21, a support pillar 23 mounted on the base 21 and two extending support arms 12, 13 which extend from the support pillar 23 over the base 21, a first support arm 13 extending from the top of the support pillar 23 and a second support arm 12 extending from the support pillar 23 about two-thirds of the way from the first support arm 13 to the base 21 in the particular of the system 1 configuration illustrated in FIG. 1.

The system 1 further includes a cell chamber 11 mounted on the second support arm 12 and an electromagnetic probe 19 including an electromagnet extending downwards from the first support arm 13 towards the cell chamber 11.

The system 1 further includes a lens system 15 mounted on the base 21 directly below cell chamber 11 which operates in conjunction with a light source (not visible) which is mounted within the first support arm 13 for imaging the contents of the cell chamber 11, the second support arm 12 being configured to enable imaging of the cell chamber 11, having a suitably positioned aperture or similar.

The first support arm 13 is manually movable with respect to the support pillar 23 for adjusting the position of the electromagnetic probe 19 with respect to the cell chamber 11, i.e. adjusting a distance of the electromagnetic probe 19 from the cell chamber 11 or an angle of the electromagnetic probe 19 with respect to the cell chamber 11.

A controller 17 mounted in the support pillar 23 enables control of the lens system 15 and light source and switching on and off of the electromagnetic probe 19.

In use, at least one cell-culture device or, equivalently, cell-culture platform 100 (not visible), on which cells may be cultured is positioned within the cell chamber 11. The cell culture platform 100 is deformable in response to actuation by a magnetic field and therefore the electromagnetic probe 19 is operable, when switched on via the controller 17, to actuate the deformation of the at least one cell-culture platform 100.

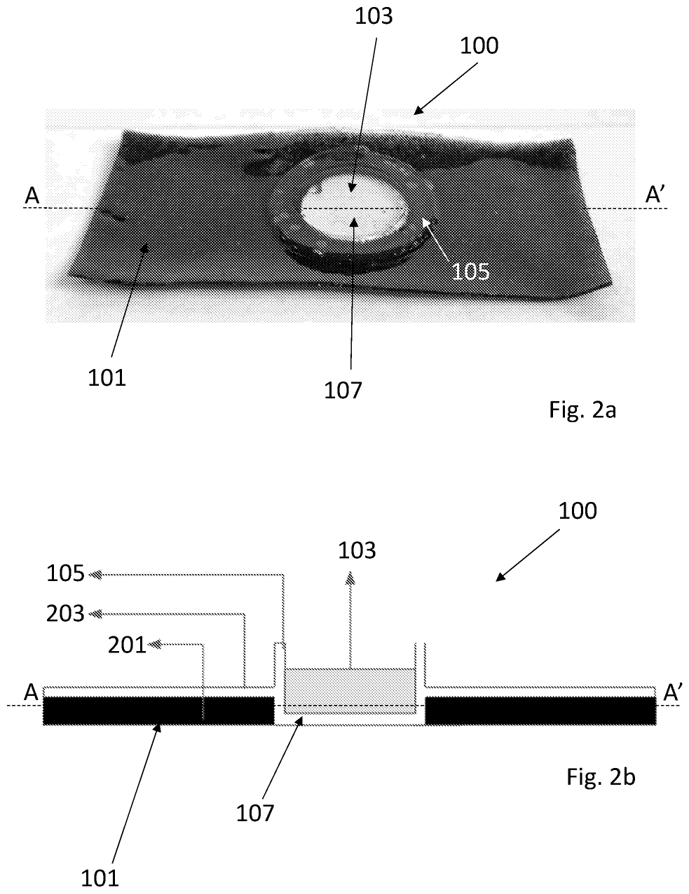

FIG. 2a is a photograph of the cell-culture platform 100 for positioning within the cell chamber 11 according to the described embodiment. The cell-culture platform 100 includes substrate 101 and a biocompatible receptacle for cell culture media. In this embodiment, the substrate 101 is elongate and substantially planar and the biocompatible receptacle comprises a circular well 103 positioned approximately centrally in the substrate 101, with the substrate 101 encircling the well 103 in a plane of the substrate 101, i.e. in the plane comprising an imaginary axis AA′. The circular well 103 includes a base 107 and a circumferential raised wall 105, attached to the substrate 101 and separating the substrate 101 from the well 103. In the described embodiment, the cell-culture platform 100 is integrally formed as a multi-layer composite material based on an elastomeric material in the form of a transparent silicone rubber, and the substrate 101, wall 105 and base 107 (and hence cell culture well 103), are all operable to undergo mechanical deformation e.g. stretching and/or compression. Thus, the cell-culture platform 100 may be considered as having a unitary structure. Note that a resting—i.e. undeformed-state of the composite is illustrated in FIG. 2a. In this state, the substrate 101 is substantially opaque (for reasons which will become apparent below), while the base 107 is substantially transparent enabling imaging of cells cultured within the well 103.

FIG. 2b is a schematic illustration of a cross section of the cell-culture platform 100 illustrating the detailed structure of the composite material according to the described embodiment. The substrate 101 includes two layers 201, 203: a deformation actuator layer 201 and a transparent upper layer 203 of the silicone rubber arranged on the deformation actuator layer 201.

In the described embodiment, the deformation actuator layer 201 consists of a layer of a magnetic material. The magnetic material includes a substantially homogenous dispersion of magnetic particles, in the form of iron filings according to the described embodiment, in the elastomeric material. As used herein, a substantially homogenous dispersion, or equivalently an even distribution, means that no clumps of magnetic particles are discernible by visual inspection of the magnetic material.

For example, the weight of the iron filings may amount to about 60% of the weight of the transparent silicone rubber in the magnetic material according to the described embodiment, i.e. for every 100 g of the transparent silicone rubber in the magnetic material, there is about 60 g of iron filings and the total weight of the magnetic material is therefore about 160 g. In this ratio, the quantity of iron filings in the transparent silicone rubber may render the magnetic material substantially opaque in the resting (i.e. undeformed) state of the platform 100, despite the transparency of the transparent silicone rubber.

The base 107 includes a single layer of the transparent silicone rubber of approximately the same thickness as the upper layer 203 (for example, about 200 μm), hence the well 103 is depressed in profile relative to the substrate 101. The wall 105 is formed from a single layer of the silicone rubber and is in contact with and extends perpendicular to deformation actuator layer 201, upper layer 203 and base 107, and separating the base 107 from the deformation actuator layer 201. The wall 105 extends above the two layers of the substrate 101, forming a raised embankment around the well 103.

The transparent upper layer 203 creates an additional layer of biocompatible inert material over the deformation actuator layer 201 and additionally may help to provide a substantially uniformly transparent platform for visualisation of cells in the well 103.

An exemplary thickness of the upper layer 203 and the base 107 is from about 80 μm to about 100 μm. An exemplary thickness of the wall 105 is about 2.5 mm. An exemplary thickness of the deformation actuator layer 201 is from about 500 μm to about 2500 μm. In an example, the well 103 has a diameter of about 15 mm.

In use, cells may be seeded into the well 103 and an applied magnetic field generated from electromagnetic probe 19 to actuate the iron filings in the magnetic material forming the deformation actuator layer 201, causing deformation of the magnetic material and consequently deformation of the well 103 and the cells seeded into the well 103. Stretch and/or compression of the seeded cells may therefore be achieved via the application of a magnetic field. The arrangement of the magnetic material around and to enclose the well 103, means that deformation of the well 103 may be achieved in any direction around the platform 100.

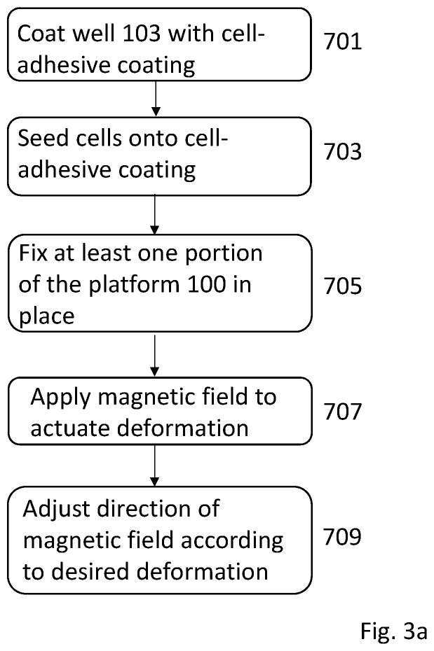

A detailed exemplary method of stretching and/or compressing cells using the system 1 is illustrated in FIG. 3a. The method comprises steps 701 to 709.

In step 701, one or more inner surfaces of the well 103 (for example, the base 107) of the cell-culture platform 100 is coated with a tissue environment mimetic material which acts as cell-adhesive or cell-binding coating to ensure that cells are fixed in place in the well 103. Examples of suitable cell-binding coatings include Fibronectin, collagen and laminin.

Cells are then seeded onto the coating and into the well 103 in step 703.

In step 705, the cell-culture platform 100 is inserted into the cell chamber 11 and is fixed at one location, for example by clamping the platform at one end.

In step 707, the electromagnetic probe 19 is switched on and the cell-culture platform 100 is thus subjected to an applied magnetic field, for example perpendicular to the plane of the deformation actuator layer 201, i.e. perpendicular to the imaginary axis AA′ shown in FIG. 2a and FIG. 2b,

Attraction between the iron filings embedded in the magnetic material of the deformation actuator layer 201 and the magnetic field causes deformation of the deformation actuator layer 201, which, comprising silicone rubber, is elastomeric. Because the deformation actuator layer 201 is attached to the wall 105, which itself is attached to the base 107 (both of which are themselves formed from silicone rubber), deformation of the deformation actuator layer 201 in turn actuates deformation of the wall 105 and base 107 along a same deformation axis, i.e. deformation of the entire well 103 and consequently compression and/or stretching of the cells seeded in the well 103 along the deformation axis.

The deformation of the cell culture platform 100 using the electromagnetic probe 19 of FIG. 1 is illustrated schematically in FIG. 3b, showing the platform 100 being deformed towards the electromagnetic probe 19.

In step 709, adjustment in the position of the electromagnetic probe 19, via adjustment of the support arm 13 is performed in order to achieve the desired stretch and/or compression of the cells adhered to one or more inner surfaces of the well 103 via the coating, if required, for example, changing the angle of the electromagnetic probe 19 relative to the platform 100 or changing the size of the adjustable gap electromagnetic probe 19 and the cell chamber 11. The relative transparency and small thickness (for example, less than about 100 μm) of the base 107 enables imaging of the cells using the lens system 15.

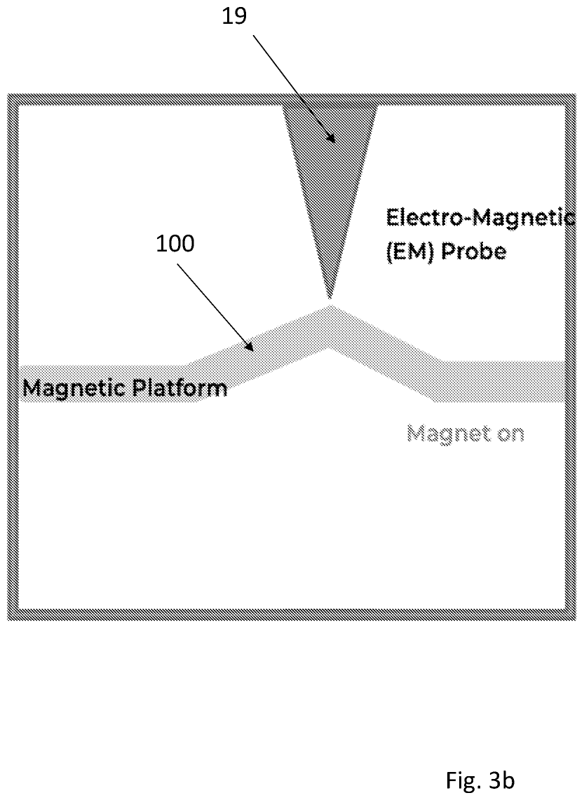

An exemplary fabrication method of the cell-culture platform 100 according to the described embodiment is illustrated in FIG. 4. The method comprises steps 301 to 309.

In step 301, liquid silicone rubber is mixed with, for example, iron filings until the filings are substantially evenly distributed and weight of the iron totals about 60% of the weight of the silicone rubber alone. The mixture is then cured in step 302 to form the deformation actuator layer 201, for example by spin casting the mixture onto a salinized coverslip and baking.

In step 303, a circular portion, or through-hole, is removed from the deformation actuator layer 201 formed in step 302, for example using a biopsy punch to form a cavity for the well 103.

In step 305, the deformation actuator layer 201 is coated with liquid silicone rubber to form both the upper layer 203 and base 107, for example by repositioning the deformation actuator layer 201 on a silanized glass cover slip and spin coating the cover slip with the liquid silicone rubber.

In step 307, liquid silicone rubber is cured in the form of a single layer, for example, by spin casting onto a salinized coverslip and baking. A ring is then cut from the formed layer, for example by cutting a circle using a biopsy punch and removing the centre of the circle, thus forming the wall 105.

In step 309, the wall 105 formed in step 307 is positioned over the circular cut out on the coated deformation actuator layer 201 produced in step 305. Finally, the coating applied in step 305 is cured.

Although a particular method of fabrication of the magnetic material and cell-culture platform 100 is described above it is envisaged that the above steps may be performed in a different order or two or more steps may be performed concurrently. Further, it is envisaged that other methods of fabrication of the magnetic material and/or cell-culture platform 100 could be employed. For example, it is envisaged that the deformation actuation layer 101 may be formed within a mould in which variation, the cavity for the well 103 may be formed by means of a pillar comprised within the mould, rather than by cutting out a though hole as described above. The method of fabrication may be chosen according to context and intended use.

As described above, the design of the cell-culture platform 100, according to the described embodiment, in particular the inclusion of the magnetic material forming the deformation actuator layer 201, enables cell compression and stretching. Upon the application of an external magnetic field, the magnetic material forming deformation actuator layer 201 acts as an integrated actuator to deform the flexible composite.

Complementary to its biocompatible nature arising from the use of a silicone rubber as the base elastomer for the composite in combination with the tissue environment mimetic material which acts as a cell-binding coating, the composite includes a depressed well 103 with heightened embankments formed from wall 105 to contain cell culture media for cell seeding.

The design of the cell-culture platform 100 may enable extensive deformation of cells cultured in the well 103 as well as precise control over the direction of the deformation by altering the direction of the applied magnetic field with respect to the position of the cell-culture platform 100, for example by adjusting the position of the electromagnetic probe 19. This may enable flexibility in the control of force parameters of mechanobiological experiments, thereby potentially enabling research scientists to have greater freedom in manipulating the extent and direction of spatio-temporal stretch to better mimic the in-vivo conditions of their chosen cell line or, for replicating precise environmental conditions to further their research.

In particular, the dispersion of iron filings, which function as induced bar magnets (i.e. their magnetism is induced by an applied magnetic field), arranged in an elastomeric material provides a magnetic material which can be precisely deformed in any direction according to the direction and strength of an applied magnetic field. By enclosing the well 103 in the magnetic material, as in the arrangement of the platform 100, the magnetic material thus enables the well 103 to be deformed in any direction.

A first prototype of the cell-culture platform 100 according to the described embodiment was fabricated and experiments performed to demonstrate specific advantages of the magnetic material and the cell-culture platform 100 according to the described embodiment.

Specifically, a first prototype cell-culture platform according to the described embodiment was produced over a three-day process in accordance with the method of FIG. 4. The liquid silicone rubber base material for the composite was purchased from WACKER™ Silicones (Silpuran™ 6000/05). Fibronectin utilised in cell seeding was purchased from Merck Pte Ltd. 700 mm×200 mm neodymium magnets were employed.

Glass coverslips were first silanized on the first day. 10 microlitres of silane were added with the coverslips and kept overnight in the vacuum of a desiccator, under a chemical hood.

On the following day, a clear embankment ring to form wall 105 and iron-embedded deformation actuator layer 201 were fabricated. The clear embankment ring was fabricated by mixing about 1 g Part A and about 1 g Part B of the liquid silicone rubber for each coverslip. The mixture was mixed thoroughly and degassed for at least about 15 minutes before spin casting at about 500 rpm for about 30 seconds on the silanized 75 mm×50 mm×1 mm glass coverslip. The coverslip and coating were then baked in a preheated oven at about 165° C. for 5 minutes. Using a 20 mm biopsy punch, the circular blade was carefully rotated through the clear, baked elastomer. The 15 mm biopsy puncher was centralised on the cut out and the process was repeated to create the clear embankment ring for use on the next day.

For the iron-embedded deformation actuator layer 201, about 1 g Part A and about 1 g Part B of the liquid silicone rubber was mixed. Iron filings totalling about 60% by wt. of the liquid silicone rubber alone were added and the mixture was mixed thoroughly until no clumping of the iron filings was visible upon visual inspection and degassed for 1 hour before spin casting at about 1000 rpm for about 30 seconds on the silanized 75 mm×50 mm×1 mm glass coverslip. The coverslip and coating were baked in a preheated oven at about 165° C. overnight.

On the third day, the baked iron-embedded deformation actuator layer 201 was removed from the oven. The deformation actuator layer 201 was carefully peeled off the glass coverslip and a 15 mm biopsy punch was employed to carefully rotate the circular blade through the layer. The deformation actuator layer 201 was repositioned over the same coverslip. About 1 g Part A and about 1 g Part B of the liquid silicone rubber were thoroughly mixed for each coverslip and degassed for at least about 15 minutes. The degassed mixture was spun over the baked iron-containing deformation actuator layer 201 at about 2000 rpm for about 1 minute before tweezers were employed to position the clear embankment ring over the about 15 mm circular cut out. The arrangement was baked in a preheated oven at about 165° C. for about 5 minutes.

Thus, the dimensions of the completed composite were approximately 75 mm×50 mm with a circular well of diameter of about 15 mm.

The completed composite was then kept in a covered petri-dish until treated in a plasma cleaner for a total of about 15 minutes. The composite was then subjected to UV treatment in a biosafety cabinet for about 30 minutes. About 50 μl of about 10% fibronectin was then added to the depressed well 103 and left overnight in an incubator.

For cell seeding, GFP-empty vector and RFP-Lifeact transfected Hela cells were prepared in EMEM cell culture media with about 10% Fetal Bovine Serum. Prior to cell seeding, cells were incubated in dishes at about 37° C. in a humid atmosphere with about 5% CO2, and the medium was replaced every 2 days. To seed the HeLa on the fibronectin-coated well 103, the media was removed and cells were rinsed with DPBS-2 ml. Subsequently, the DPBS was completely removed and about 2 ml of 1X Trypsin was added for about 5 minutes before cells were centrifuged at about 1500 rpm for about 3 mins. Fresh media was added and the cells were directly pipetted onto the depressed culture well 103 on the elastomer and incubated. Images of transfected HeLa were acquired using a 40× aperture on a Nikon CSU-W1 confocal microscope connected to a camera. Brightness and contrast were adjusting in post-processing of the images.

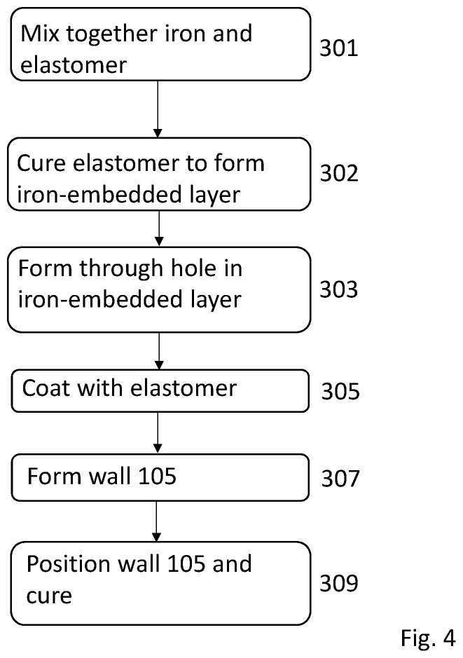

Fluorescent images of the Hela cells were captured at three wavelengths—DIC, GFP and RFP. FIG. 5 shows one of the images with an HeLa cell 501 visible, the image being captured at GFP. The successful culture of the cells on the fibronectin-coated surface of the culture well 103 demonstrates the biocompatibility of the composite produced as described above.

Following cell-culture, the first prototype was subjected to a manual replication of cyclic, uniaxial mechanical stretch. One end of the composite was fixed by attaching a 3D printed clasp to the first prototype and the other was deformed by the manual exertion of force. The setup was positioned next to a ruler for quantification of the extent of stretch achieved.

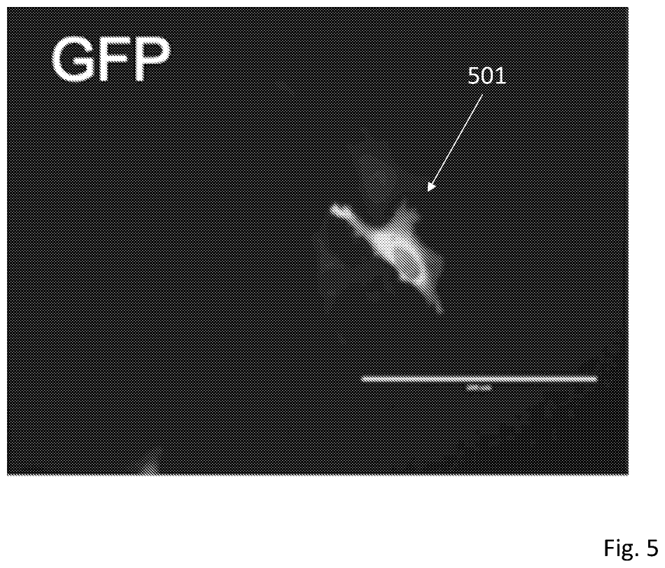

FIG. 6 shows a photograph showing mechanical stretching of the first prototype composite. As shown in the photograph, the composite was able to undergo a large deformation without debonding upon induced uniaxial mechanical stretch. All of the component layers remain bonded with no signs of tears or peeling layers.

Finally, fixed strength neodymium magnets were used to induce a permanent magnetic field. One end of the composite was fixed and the other was left unrestricted to be deformed by magnets which were positioned a fixed distance away. Whilst maintaining relative distance from the composite, the angle of the magnets was manually altered to alter their magnetic field and induce a response from the integrated deformation actuator layer 201 of the composite.

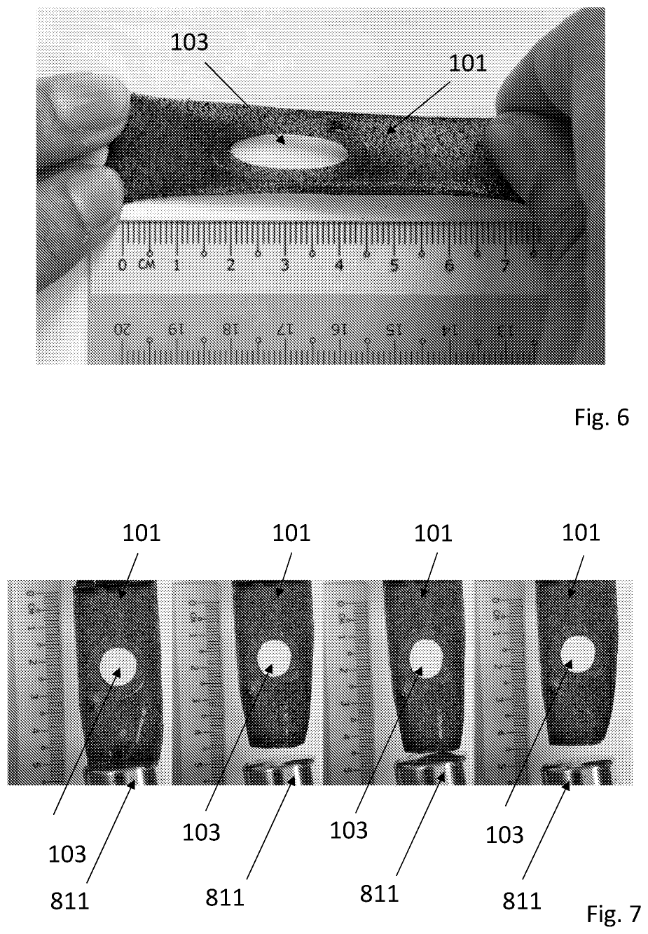

FIG. 7 shows a series of four photographs illustrating the deformation induced by the iron-embedded deformation actuator layer 201 acting as integrated actuators upon the application of the magnetic field by a neodymium magnet 811. The flexible composite was responsive to changes in the magnetic field caused by slight rotations of the permanent neodymium magnets 811.

As demonstrated by the above experimental results, the flexible composite according to the described embodiment may be capable of handling large deformations of a biocompatible cell-culture substrate, such as well 103, hence expanding the range of in-vitro conditions which may be replicated in the lab. This may grant greater freedom to researchers who might have been restricted by the limitations of other, less-flexible elastomers. In particular, the composite may allow for stretch deformations beyond 100%, potentially improving the capability to mimic in vivo conditions such as those experienced by epidermal and endothelial tissues.

Further, the integrated actuators of the cell-culture platform 100 in the form of evenly dispersed iron-filings may enable controlled deformations of any precise direction at will, thus potentially enabling fine control to induce specific, spatial-temporal deformation of the cultured cells by manipulating an external magnetic field.

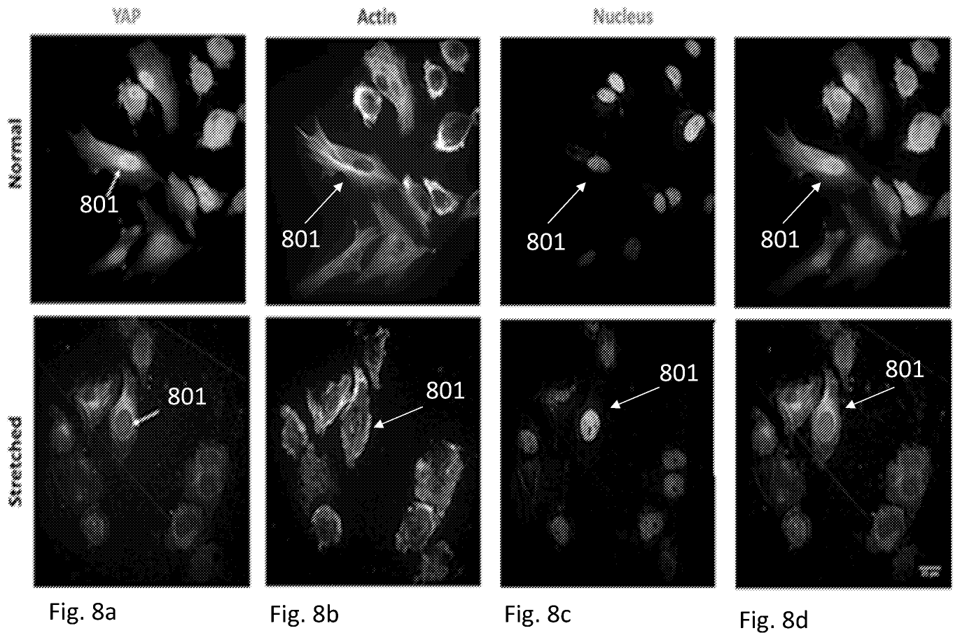

Cell binding to the well 103 under stretch was tested experimentally. Cancerous HeLa cells were seeded into the well 103 as described above and the prototype was subjected to about 30 minutes of around 20% stretch using a combination of physical and magnetic stretching. The results are shown in FIGS. 8a to 8d with the same HeLa cell 801 labelled in each image for clarity. The upper images show the HeLa cell 801 prior to stretching and the lower images show the HeLa cell after an around 20% stretch of the prototype platform for about 30 minutes. Confocal imaging through a spinning disk microscope was employed to obtain images of different cell features, with biochemical sensor YAP shown in FIGS. 8a, Actin shown in FIGS. 8b and nuclei shown in FIGS. 8c. FIG. 8d shows an overlay of all three images shown in FIGS. 8a-8c.

As is illustrated in FIGS. 8a to 8d, the stretch led to the translocation of a biochemical YAP from the nucleus to the cytoplasm and caused actin cytoskeleton breakdown in the HeLa cell 801. The results show that the cells survive well on the material of the prototype and remain attached after stretching, including to the point of undergoing changes in cellular morphology and the biochemical sensor YAP.

As further demonstrated, the fabrication protocol according to the described embodiment may enable the consistent adhesion of layers, allowing for extensive deformations beyond about 100% stretch without debonding of the layers or compromising on the integrity of the composite. The protocol is also relatively straightforward and fast.

Further prototypes of the cell-culture platform 100 according to the described embodiment were fabricated using the same three-day process and quantities described above for the first prototype and experiments performed to further demonstrate the mechanical properties of the magnetic material and the cell-culture platform 100 according to the described embodiment as well as its ability to stimulate mechanical response at both cellular and tissue levels.

A platform according to the design of FIGS. 2a and 2b was also fabricated using only polydimethylsiloxane (PDMS) for all elastomeric layers, i.e. the platform was fabricated without the inclusion of iron for comparison.

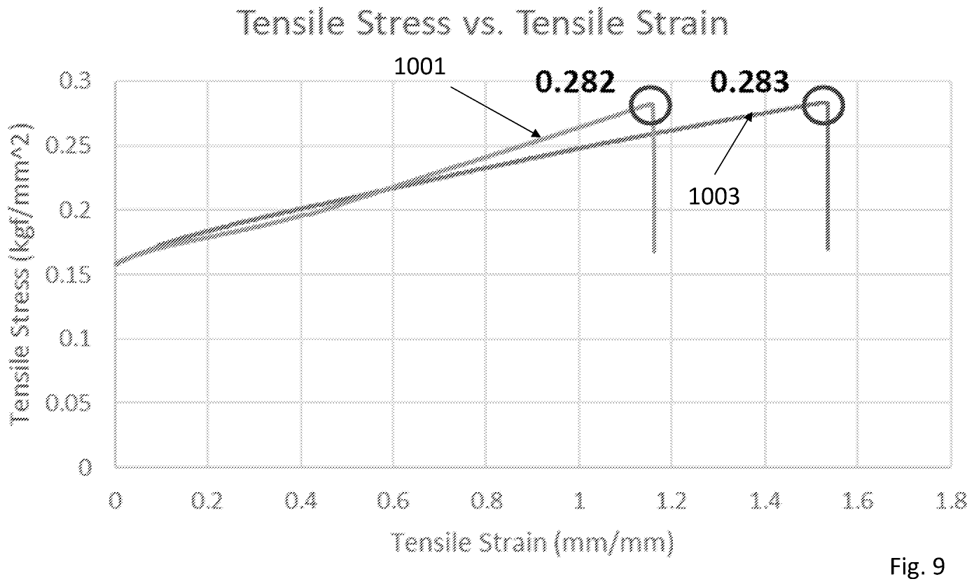

FIG. 9 shows two measurements of tensile stress as a function of tensile stress obtained for two of the further prototypes according to the described embodiment, with results for a first further prototype shown by line 1001, and the results for a second further prototype according to the described embodiment shown by line 1003 As shown in FIG. 9, the tensile strength of the platform 100 according to the described embodiment was found to be 0.283 MPa for the first further prototype and the tensile stress for the second further prototype was found to be 0.282 MPa, thus there was consistency between the two further prototypes.

Experiments were repeated with more of the further prototypes according to the described embodiment and the Average Maximum Tensile Stress for the further prototypes was determined to be 2.77 MPa (with a corresponding average tensile strain of 1.35) compared with 2.24 MPa average tensile stress for the PDMS prototype. The average Young's/Elastic Modulus of the tested further prototypes was determined to be 2.05 MPa compared with 0.36-0.87 MPa for the PDMS prototype.

Thus, the platform 100 and magnetic material according to the described embodiment may have comparable or superior properties relative to PDMS.

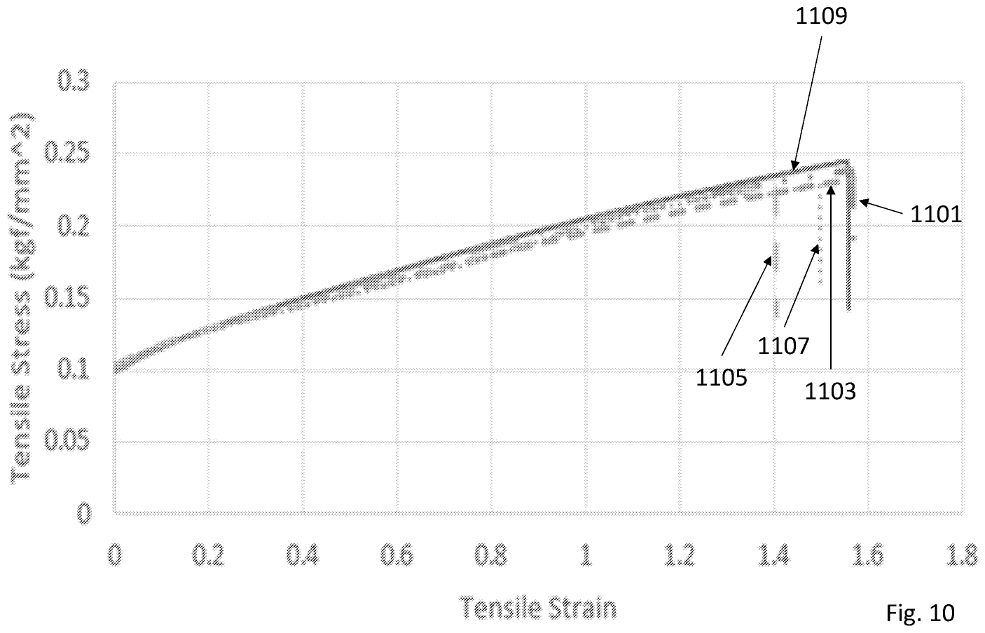

The temperature effect on the tensile stress/strain properties of the platform 100 was investigated experimentally and the results are illustrated in FIG. 10. Specifically, four further prototype platforms fabricated as described above for the first prototype were subjected to different temperatures relevant to research settings, specifically room temperature (shown by dot-dash line 1101), 37° C. (shown by short dashed line 1103), −20° C. (shown by long dashed line 1105), 4° C. (shown by dotted line 1107) and at the datum temperature (shown by solid line 1109).

The average elastic modulus was also calculated for each temperature and found to be approximately 1.54 MPa at the datum temperature, approximately 1.50 MPa at room temperature, approximately 1.40 MPa at 37° C., approximately 1.50 MPa at 4° C. and approximately 1.60 MPa at −20° C. Thus, the overall range was from approximately 1.40 MPa to approximately 1.60 MPa. Thus, there was no significant change in the tensile strength over a range of temperatures which are typically employed in research settings, indicating a potential robustness of the platform 100.

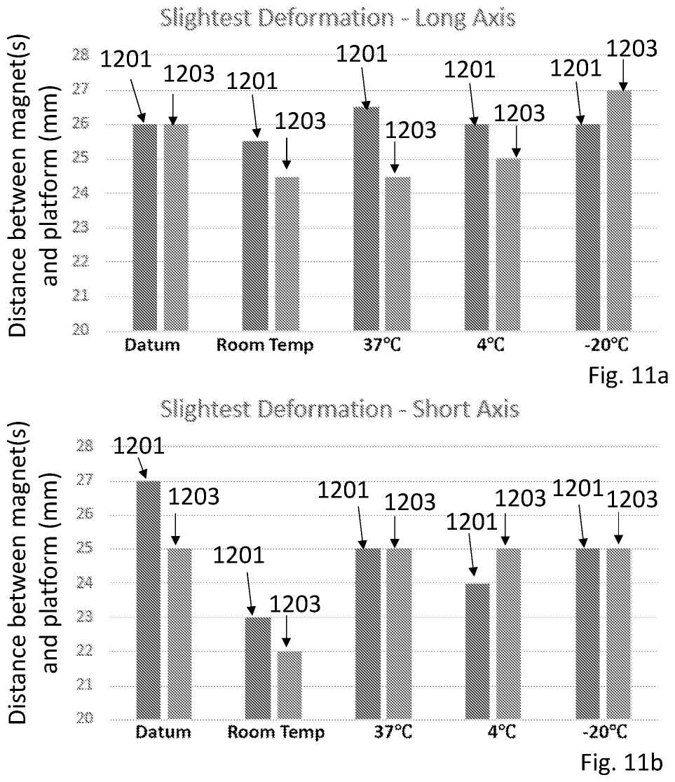

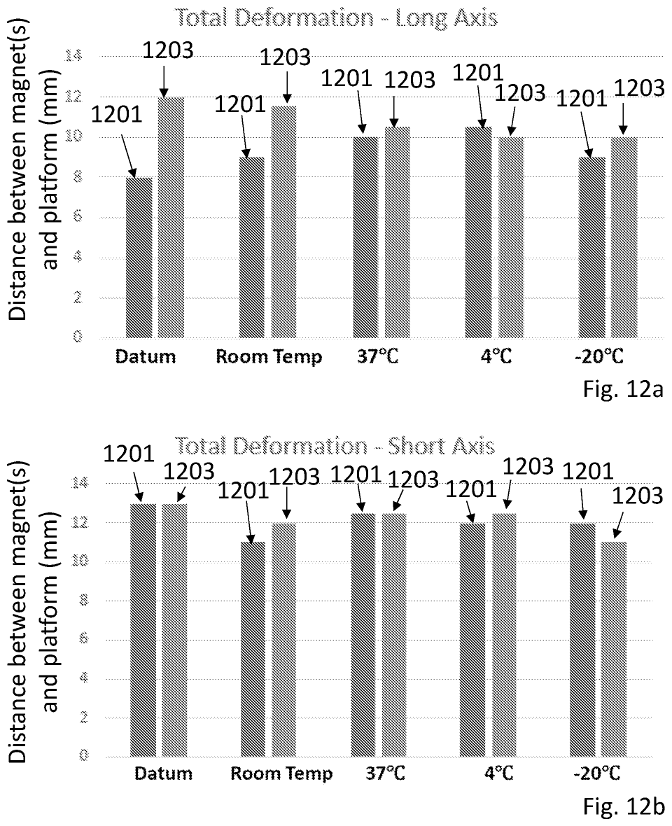

The sensitivity to magnetic influence of the each of the further prototype platforms for which results are shown in FIG. 10 was also tested and the results are shown in FIGS. 11 and 12. Specifically, FIGS. 11a and 11b show the maximum distance between an approximately 1.05 Tesla magnet and the corresponding further prototype platform at each temperature at which any deformation was observed. FIG. 11a illustrates the results obtained along the long axis of the platform 100 (i.e. along the imaginary axis AA′, as shown in FIGS. 2a and 2b) and FIG. 11b illustrates the results obtained along the short axis of the platform 100 (i.e. alone the axis perpendicular to the imaginary axis AA′, in the plane of the platform 100). In both cases results were obtained before tensile testing (indicated by bars 1201) and after tensile testing (indicated by bars 1203).

In contrast, FIGS. 12a and 12b show maximum distance between the magnets and the platform 100 at which it was possible to achieve total deformation of the corresponding prototype platform, with FIG. 12a illustrating the results obtained along the long axis of the platform 100 (i.e. along the imaginary axis AA′, as shown in FIGS. 2a and 2b) and FIG. 11b illustrating the results obtained along the short axis of the platform 100 (i.e. along the axis perpendicular to the imaginary axis AA′, in the plane of the platform 100). Again, in both cases results were obtained before tensile testing (indicated by bars 1201) and after tensile testing (indicated by bars 1203).

Again, there was no significant deviation between prototypes at different temperatures observed and the sensitivity to magnetism remained broadly consistent before and after tensile testing indicating that the magnetic responsiveness is robust.

The ability of the platform 100 to stimulate mechanical response at both the cellular and tissue levels was also tested experimentally. Specifically, a monolayer tissue of mammalian cell line MDCK expressing YAP-GFP was seeded into the well 103 of a further prototype. An approximately 1.05 T magnet was employed to stretch the further prototype platform to about 150% of its original length for four hours, before the magnet was removed to allow the further protype platform to relax back to its original length. Time lapsed snapshot images of YAP nuclear translocation were obtained both before and after stretching. It was observed that that greater amounts of YAP gradually translocated into the nuclei of the tissue after stretching.

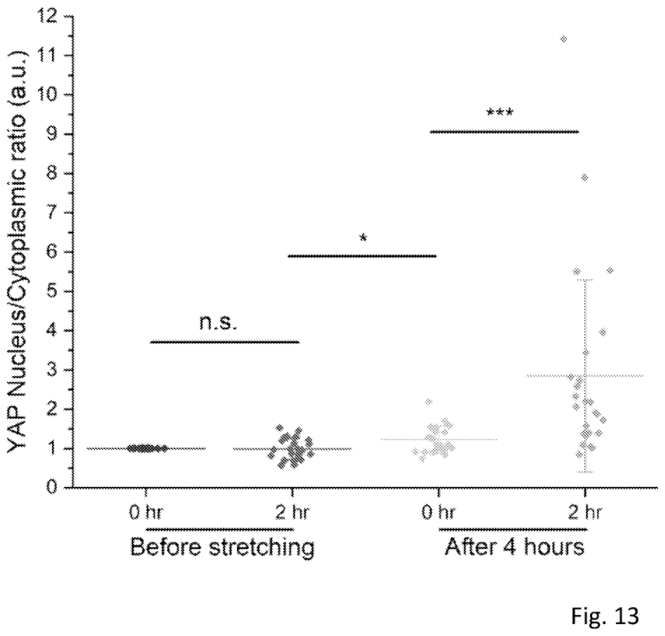

The YAP nucleus/cytoplasmic GFP intensity ratio over time before and after four hours of stretching for n=25 cells is illustrated as a scatter plot in FIG. 13, with all nucleus/Cytoplasmic YAP intensity ratios for all groups normalized to the corresponding ratio in 0 hr before stretching. Before stretching, the nucleus/cytoplasmic ratio after 2 hours was not significant (n.s., P=0.198, obtained from Mann Witney test) when compared with the ratio at 0 hours. In contrast, following 4 hours of stretching the nucleus/cytoplasmic ratio 2 hours after stretching showed a relatively higher nucleus/cytoplasmic ratio (*** P=1.04×10⁻⁴) when compared with 0 hours after stretching. The ratio immediately following 4 hours of stretching (i.e. at 0 hours after stretching) itself also showed a somewhat higher nucleus/cytoplasmic ratio (* P=1.0161) when compared with theratio before stretching (at 0 hours).

YAP nuclear translocation induced by stretch related activities is known to occur in-vivo (see for example Lin Grimm, Hiroyuki Nakajima, Smrita Chaudhury, Neil I Bower, Kazuhide S Okuda, Andrew G Cox, Natasha L Harvey, Katarzyna Koltowska, Naoki Mochizuki, Benjamin M Hogan (2019) Yap1 promotes sprouting and proliferation of lymphatic progenitors downstream of Vegfc in the zebrafish trunk eLife 8:e42881, https://elifesciences.org/articles/42881 which concerns such processes in zebrafish) therefore the platform 100 according to the described embodiment may enable replication of in-vivo processes.

The described embodiment should not be construed as limitative. For example, although the magnetic material is described as being employed in the cell-culture platform 100, it is envisaged that the magnetic material could be employed to enable controlled deformation of a structure or device using a magnetic field in other platforms and devices, including in fields unrelated to cell-culture or biological applications.

Although iron filings embedded in a silicone rubber are described above, it will be appreciated that other ferro-, ferric- or para-magnetic materials could be employed in place of iron. Likewise, permanent magnetic particles rather than particles acting as induced magnets could be employed. In particular, ferromagnetic materials could be employed as the magnetic particles in the magnetic material according to embodiments, in the form of filings or in another particulate form. Suitable alternative magnetic particles include but are not limited to those including Cobalt, Nickel, Gadolinium, Dysprosium, Permalloy and Wairakite

The total weight of the magnetic particles may be greater or lesser than about 60% of weight of the elastomeric material (excluding the magnetic particles) in the magnetic material, for example from about 40% to about 80%, particularly from about 55% to about 65% of the weight of the elastomeric material. In general, the lower the quantity of magnetic particles relative to the weight of elastomer, the greater the stretching achievable with the using the magnetic material, however, the sensitivity to magnetic fields will be lower. A higher percentage of magnetic particles relative to the weight of elastomer results in a greater magnetic response but may reduce the amount by which the platform can be stretched. For example, when the total weight of the magnetic particles is about 60% of the weight of the elastomeric material then the elongation at break may be about 250%. When the total weight of the magnetic particles is about 40% of the weight of the elastomeric material then the elongation at break may be about 350%.

In particular, the total weight of the magnetic particles in an amount of about 60% of the weight of the elastomeric material, as in the described embodiment, may achieve a good balance between the extension of the platform which is achievable by stretching and the responsiveness of the platform to magnetic fields.

It is envisaged that any elastomeric material (including elastomeric materials other than silicone rubber) having biocompatibility (i.e. not containing any material which could be harmful to biological material) and having high elasticity could be employed in one or more of the layers of the composite material, including the magnetic material comprised in the deformation actuator layer 201.

In particular, it is envisaged that an elastomeric material having sufficient elasticity such that, when employed in the magnetic material, the elongation at break of the magnetic material is from greater than about 100% when determined using ISO 37 type 1, particularly about 100% to about 400%, more particularly about 200% to about 300%, further particularly about 250% could be employed. The above elongation at break may be achieved when the total weight of the magnetic particles in the elastomeric material is in the range from about 40% to about 80% of the weight of the elastomeric material, such as from about 55% to 65%, such as about 60%.

In particular, an elastomeric material having low brittleness is employed, for example an elastomer which enables an elongation of at least about 100% when determined using ISO 37 type 1 with minimal crack formation and propagation, particularly at least about 250% with minimal crack formation and propagation.

Although a transparent elastomer is employed in the magnetic material of the described embodiment, it is envisaged that non-transparent elastomers could also be employed.

The magnetic material may not be homogeneously or substantially homogeneously distributed throughout one or more layers of the composite, i.e. the magnetic material may be heterogeneously, or unevenly distributed. For example, the magnetic material, such as iron filings, may be more concentrated in a particular area of an elastomeric layer, for example in order to allow stretch or compression in a particular direction, i.e. a specific area distribution.

The distribution of magnetic particles could be embedded on the surface of the composite or a layer of the composite, as opposed to embedded within a layer.

The magnetic particles may be larger or smaller than iron filings. For example, the size of the magnetic particles in one dimension may lie in a range from about 0.05 mm to about 0.15 mm.

It will be appreciated that the thicknesses of the layers could be varied according to requirements. For example, the thickness of the deformation actuator layer 201 may lie in a range from about 500 μm to about 2500 μm. Likewise, it will be appreciated that although dimensions of prototype platforms according to the described embodiment are given above, the dimensions of the platform may be varied according to requirements.

The cell-culture platform 100 may not comprise a composite material, instead being formed of separate, attached components. Although the cell culture media receptacle, such as the well 103, should be biocompatible, the other components in the cell-culture platform 100 may not be.

Although the well 103 is described as being formed within the substrate 101, with the deformation actuator layer 201 surrounding the well 103, the deformation actuator layer could instead be incorporated into a part of the well 103 or other receptacle for cell-media, for example the wall 105, or base 107. The deformation actuator layer 201 may not completely surround the well 103, being present only on one side of, or in a number of discrete locations around, the well 103.

It is envisaged that the wall 105 may not be present, or may not extend above the substrate 101.

It is envisaged that cell culture platform 100 could be employed for the culture of any type of cell, not only those which are susceptible to undergoing deformation in the mammalian body. Examples of cells which could be cultured on cell culture platform 100 include, but are not limited to cells of connective, muscle, epithelial, and nervous tissues.

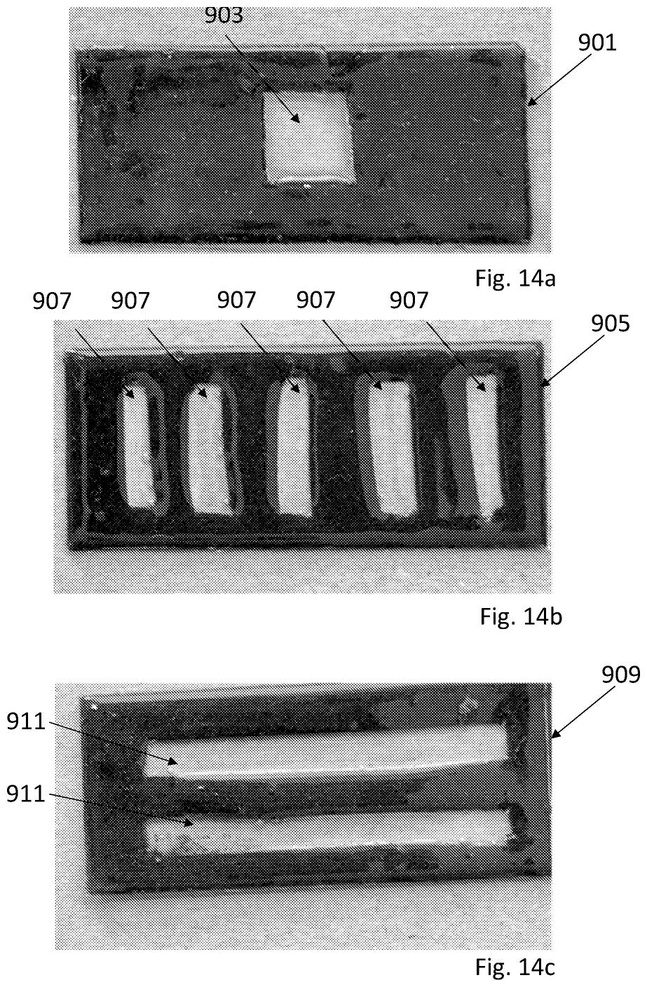

Although, in the described embodiment, the cell culture platform 100 includes a single, circular well 103, it is envisaged that the cell culture platform 100 could include a plurality of receptacles for cell culture media and the receptacles for cell-culture media may not be circular, having, for example, a rectangular form. Three examples of alternative cell-culture platform designs according to different embodiments having differently shaped receptacles are shown in FIGS. 14a-14c.

FIG. 14a is a plan-view photograph of a cell-culture platform 901 according to a second embodiment having a single, substantially centrally positioned square-shaped cell culture media receptacle 903. Otherwise, the cell-culture platform 901 is substantially identical to that of the described embodiment shown in FIGS. 2a and 2b.

FIG. 14b is a plan-view photograph of a cell-culture platform 905 according to a third embodiment. The cell-culture platform 905 has a substantially rectangular form and includes five substantially parallel cell culture media receptacles 907. Each of the cell culture media receptacles 907 is approximately rectangular in shape, extending parallel to the direction of the shorter edge of the cell-culture platform 905. Otherwise, the cell-culture platform 905 is substantially identical to that of the described embodiment shown in FIGS. 2a and 2b.

FIG. 14c is a plan-view photograph of a cell-culture platform 909 according to a fourth embodiment. The cell-culture platform 909 has a substantially rectangular form and includes two substantially parallel cell culture media receptacles 911. Each of the cell culture media receptacles 911 has an approximately rectangular elongate shape, extending parallel to the direction of the longer edge of the cell-culture platform 911, i.e. oriented perpendicular to the cell culture media receptacles 907 of the embodiment of FIG. 14b. Otherwise, the cell-culture platform 905 is substantially identical to that of the described embodiment shown in FIGS. 2a and 2b.

Thus, the embodiments of FIGS. 14b and 14c, each having a plurality of cell culture media receptacles 907, 911, enable cells to be seeded onto different locations of each of the cell-culture platforms 905, 909 thereby enabling different amounts of stretch to be simultaneously exerted on cells cultured thereon, according to the cell culture media receptacle in which they are located.

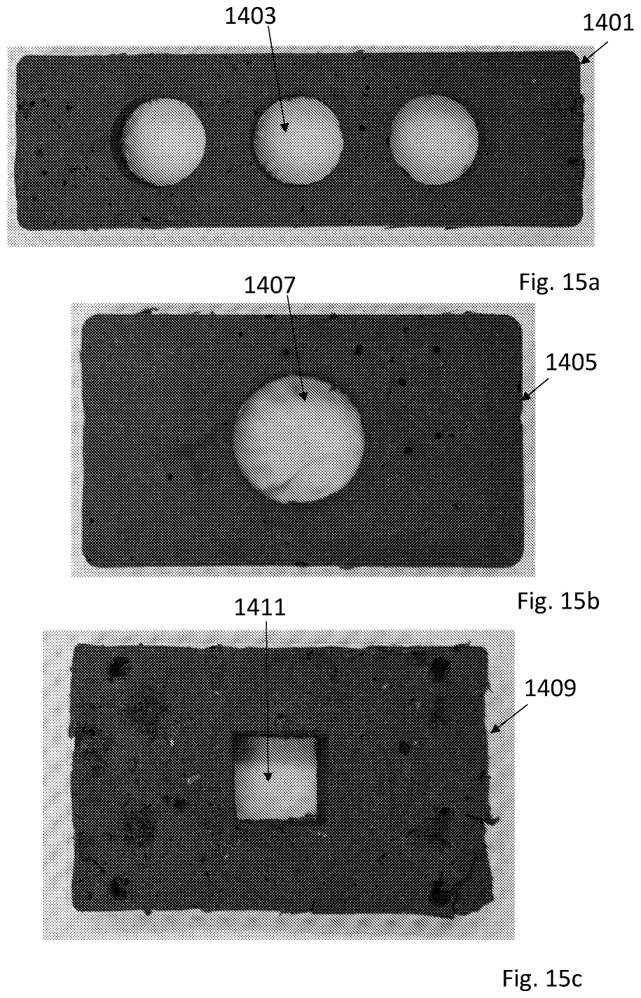

Likewise, different well configurations may be achieved via the use of moulds to produce the respective deformation actuator layers, with the different well configurations being achieved using different pillar configurations within each mould. FIGS. 15a-c illustrate photographs of three cell culture platforms 1401, 1405, 1409 formed using moulds, each with different well configurations.

Specifically, FIG. 15a shows a 140 mm×50 mm cell culture platform 1401 including three circular wells 1403 of diameter 22 mm, FIG. 15b shows an 80 mm×50 mm cell culture platform 1405 including a singular circular well 1407 of diameter 22 mm, similar to that illustrated in FIGS. 2a and 2b, and FIG. 15c shows a 55 mm×35 mm cell culture platform 1409 having a single, 12 mm×12 mm square well 1411.

Although a cell-deformation system having all the features of FIG. 1 has been described, it is envisaged that any system comprising at least one magnetic element (e.g. a permanent magnet, or an electromagnet) and a means of fixing the cell-culture platform 100 in place, in order to enable magnetic deformation of the cell-culture platform by the magnetic element could be employed with any cell culture platform 100, 901, 905, 909, 1401, 1405, 1409 according to embodiments.

Having now described the invention, it should be apparent to one of ordinary skill in the art that many modifications can be made hereto without departing from the scope of the following claims.

1. Magnetic material for a cell-culture platform, comprising:
an elastomeric material and a dispersion of a plurality of magnetic particles embedded in the elastomeric material.

2. Magnetic material for a cell-culture platform according to claim 1, wherein an elongation at break of the magnetic material is at least 100% or is from 200% to 400%.

3. (canceled)

4. (canceled)

5. Magnetic material for a cell-culture platform according to claim 1,
wherein the dispersion of the plurality of magnetic particles comprises a substantially homogenous distribution of the magnetic particles within the elastomeric material.

6. Magnetic material for a cell-culture platform according to claim 1, wherein the magnetic particles comprise magnetic-field inducible magnets, or filings, or iron filings, or one or more of Iron, Cobalt, Nickel, Gadolinium, Dysprosium, Permalloy and Wairakite.

7. (canceled)

8. (canceled)

9. (canceled)

10. (canceled)

11. Magnetic material for a cell-culture platform according to claim 1, wherein a weight of the magnetic particles is from 40% to 80% of a weight of the elastomeric material or wherein the weight of the magnetic particles is from 55% to 65% of the weight of the elastomeric material.

12. (canceled)

13. Cell-culture platform, comprising:
a biocompatible deformable receptacle for cell culture media; and
a deformation actuator attached to the biocompatible deformable receptacle, the deformation actuator comprising a magnetic material according to claim 1, and being operable to deform the biocompatible deformable receptacle in response to a magnetic field.

14. Cell-culture platform according to claim 13, wherein the biocompatible deformable receptacle comprises a further elastomeric material, and wherein the deformation actuator and the biocompatible deformable receptacle are integrally formed as a composite material.

15. Cell-culture platform according to claim 14, wherein the further elastomeric material comprises silicone rubber.

16. Cell-culture platform according to claim 13, wherein the deformation actuator encloses the biocompatible deformable receptacle and is operable to deform the biocompatible deformable receptacle along a plurality of axes.

17. Cell-culture platform according to claim 13, wherein the biocompatible deformable receptacle comprises a base which is substantially transparent.

18. A method of producing a magnetic material for a cell-culture platform, comprising: mixing a predetermined weight of magnetic particles into a quantity of a liquid elastomer to disperse the magnetic particles in the quantity of the liquid elastomer; and curing the quantity of the liquid elastomer to form the magnetic material.

19. (canceled)

20. A method of producing a magnetic material for a cell-culture platform according to claim 18, wherein the magnetic particles comprise filings, or iron filings, or one or more of Iron, Cobalt, Nickel, Gadolinium, Dysprosium, Permalloy and Wairakite.

21. (canceled)

22. (canceled)

23. (canceled)

24. A method of producing a magnetic material for a cell-culture platform according to claim 18, wherein mixing the predetermined weight of magnetic particles into the quantity of the liquid elastomer includes mixing a weight of the magnetic particles equivalent to from 40% to 80% of a weight of the quantity of the liquid elastomer or includes mixing the weight of the magnetic particles equivalent to from 55% to 65% of the weight of the quantity of the liquid elastomer.

25. (canceled)

26. (canceled)

27. A method of producing a cell-culture platform, comprising: producing a magnetic material in accordance with a method of claim 18; and attaching a biocompatible deformable receptacle for cell culture media to the magnetic material.

28. A method of producing a cell-culture platform according to claim 27, wherein attaching the biocompatible deformable receptacle for cell culture media to the magnetic material includes:
applying a further quantity of a further liquid elastomer to the magnetic material and curing the further quantity of the further liquid elastomer to form a composite material comprising the biocompatible deformable receptacle and the magnetic material.

29. A method of producing a cell-culture platform according to claim 28, wherein the further liquid elastomer comprises silicone rubber.

30. A method of producing a cell-culture platform according to claim 28, wherein
applying the further quantity of the further liquid elastomer to the magnetic material includes:
removing a portion of the magnetic material to form a through-hole in the magnetic material; and introducing the further quantity of the further liquid elastomer into the through-hole.

31. A method of deforming cells using a cell-culture platform comprising a biocompatible deformable receptacle for cell-culture media, and
a deformation actuator attached to the biocompatible deformable receptacle, the deformation actuator comprising an elastomeric material and a dispersion of a plurality of magnetic particles embedded in the elastomeric material, the method comprising:
applying a cell-binding coating to an inner surface of the biocompatible deformable receptacle;
seeding one or more cells onto the cell-binding coating; and
attracting the dispersion of the plurality of magnetic particles using an applied magnetic field to deform the biocompatible deformable receptacle and the one or more cells.

32. A method of deforming cells according to claim 31, further comprising:
adjusting an axis of deformation of the one or more cells by adjusting an angle of the applied magnetic field relative to the cell-culture platform.

33. A system for cell deformation comprising:
a magnet; and
a cell-culture platform according to claim 13.
